(19) United States
(12) Patent Application Publication
(10) Pub. No.: US 2022/0192902 A1
(43) Pub. Date: Jun. 23, 2022

(54) Post-Operative Carpal Tunnel Surgery Glove

(71) Applicant: Marvin Maaske, Rockford, IL (US)

(72) Inventor: Marvin Maaske, Rockford, IL (US)

(21) Appl. No.: 17/324,204

(22) Filed: May 19, 2021

Related U.S. Application Data
(60) Provisional application No. 63/126,813, filed on Dec. 17, 2020.

(51) Int. Cl. A61F 15/00; A61F 13/10

(52) U.S. Cl. CPC A61F 15/004; A61F 15/008; A61F 2013/00238; A61F 13/104; A61F 15/006

(57) ABSTRACT

This present patent application relates to a waterproof post-operative carpal tunnel surgery glove structured to be worn by an individual after experiencing a carpal tunnel surgery. The glove prevents a bandage and surgical stitches from getting wet for an extended period of time up to the point of the stitches being removed. The glove can be easily worn by sliding the glove over the fingers, palm, and wrist. The glove can be secured via a hook and loop fastener strap on the wrist area to conform to a wear's arm. A wearer can also adjust the fit of the glove to comfortably accommodate the hand using the hook and loop fastener strap. The glove keeps the bandage dry at all times and maximizes protection against water and cross-contamination of the bandage while hand washing, showering and the like.

### CROSS-REFERENCE TO RELATED APPLICATION

The present application claims priority to, and the benefit of, U.S. Provisional Application No. 63/126,813, which was filed on Dec. 17, 2020 and is incorporated herein by reference in its entirety.

### FIELD OF THE INVENTION

The present invention relates generally to the field of gloves. More specifically, the present invention relates to a modified glove worn over a post-operative bandage of a patient (or wearer) after undergoing carpal tunnel surgery. The glove surrounds the bandages and stitches and prevents the same from getting wet and from any cross-contamination for an extended period of time, up to the period of the removal of the stitches. The wearer can easily slide the glove on over the fingers, palm and wrist, and can secure the glove using a hook and loop fastener strap on the wrist area of the glove. The glove of the present invention provides an easy way of keeping a bandage dry while hand washing, bathing or other wet activity. Accordingly, the present invention makes specific reference thereto. However, it is to be appreciated that aspects of the present invention are also equally amenable to other like applications, devices and methods of manufacture.

### BACKGROUND OF THE INVENTION

By way of background, approximately 3 to 6 percent of adults in the general population are affected by carpal tunnel syndrome. Most individuals constantly use their hands during the day. However, individuals suffering from carpal tunnel syndrome may have pain, numbness and tingling in their fingers, hands and arms. Carpal tunnel syndrome occurs when one of the major nerves to the hand, the median nerve, is squeezed or compressed as it travels through the wrist. Carpal tunnel syndrome, if not treated timely, gets progressively worse and therefore early diagnosis and treatment are important. Various solutions are available for treating carpal tunnel syndrome, such as wrist braces and corticosteroids. In severe cases, carpal tunnel release surgery is recommended.

In carpal tunnel release surgery, the transverse carpal ligament is cut, which releases pressure on the median nerve and relieves the symptoms of carpal tunnel syndrome. An incision is made at the base of the palm of the affected hand, to allow the doctor to see the transverse carpal ligament, and once the ligament is cut, the skin is closed. After the surgery is done, the hand is wrapped with a post-operative bandage to cover the hand and protect it from dust, dirt and more, until the stitches are removed. The time period between surgery and stitch removal can be up to two weeks. The bandage and stitches must be kept dry during this healing period to prevent further injury and infection. It can be difficult for individuals to keep their bandage and stitches safe and dry for up to two weeks. During hand washing, showering and bathing, it can get incredibly difficult or nearly impossible to prevent the bandage from becoming wet. Individuals can get assistance from others while bathing, or may use a temporary covering for the bandage to keep it dry. However, none of the solutions heretofore known have proven to be effective, thereby leaving individuals frustrated, and possibly causing a delay in healing.

Therefore, there exists a long felt need in the art for a device to protect the post-operative bandage worn by individuals who have undergone carpal tunnel release surgery. There exists a long felt need in the art for a device to protect the post-operative bandages from getting wet while the individuals are using water or bathing. There is also a long felt need in the art for a solution for covering post-operative bandages and keeping them dry while bathing or hand washing. Additionally, there is a long felt need in the art for a cover for post-operative bandages which maximizes protection against water and cross-contamination of the bandage while washing the hands, showering and the like. Further, there is a long felt need in the art for a cover for a post-operative bandage which keeps the bandage and the stitches safe until the surgical stitches are removed. Furthermore, there is a long felt need in the art for a cover for a post-operative bandage that helps individuals to maintain a sanitary and dry bandage prior to surgical stitch removal. Finally, there is a long felt need in the art for a cover to be worn over a post-operative bandage, which can be comfortably worn and conveniently adjusted over the hands and wrist, as per the requirements of different wearers.

The subject matter disclosed and claimed herein, in one potential embodiment thereof, comprises a waterproof post-op carpal tunnel surgery glove structured to be worn over a hand after carpal tunnel surgery to prevent bandages and stitches from getting wet. The glove construction comprises an innermost layer, a middle layer and an outermost layer. The innermost layer provides moisture permeability; the middle layer provides waterproofing capabilities; and, the outermost layer provides dirt, dust and water repellency. The waterproof post-op carpal tunnel surgery glove is configured to be worn over a hand and covers the fingers, thumb, palm area and dorsal area of the wearer. The glove also conforms to the wrist area of the wearer in order to protect the bandage that is typically covering a portion of the palm area and the wrist area after carpal tunnel surgery. The glove includes a hook and loop fastener strap for comfortable securement over the hand and the wrist.

In this manner, the waterproof post-op carpal tunnel surgery glove of the present invention accomplishes all of the forgoing objectives, and provides a relatively safe, easy and comfortable solution for covering post-operative bandages while maintaining a sanitary and dry bandage prior to stich removal. The waterproof post-op carpal tunnel surgery glove of the present invention is also wearer-friendly, inasmuch as it enables users to easily take a bath or shower, wash hands or access water, without worrying about their post-operative bandage or stitches getting exposed to water.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a waterproof post-op carpal tunnel surgery glove structured to be worn after carpal tunnel surgery to prevent bandage and stitches from getting wet. The surgery glove comprises: an innermost layer, a middle layer and an outermost layer; the innermost layer provides moisture permeability; the middle layer provides moisture permeability and waterproofing capabilities; and the outermost layer provides dirt, dust and water repellency. The waterproof post-op carpal tunnel surgery glove is configured to be worn over a hand and covers the fingers, thumb, palm area and dorsal area while conforming to the wrist area of a wearer to protect the bandage covering, a portion of the palm area and the wrist area after carpal tunnel surgery. The glove also comprises a hook and loop fastener strap for comfortable securement over the hand and the wrist of the wearer.

In a further embodiment of the present invention, a waterproof post-op carpal tunnel surgery glove adapted for being worn by a wearer having a post-operative bandage over post-operative stitches after undergoing carpal tunnel surgery is disclosed. The surgery glove comprises a palm area, a dorsal area, a wrist area and one or more finger portions; the palm area covers the palm of the wearer; the dorsal area covers the dorsal region of the wearer; the wrist area covers the wrist of the wearer; and the finger portions cover the fingers of the wearer. The glove can be secured via a hook and loop fastener strap on the wrist area, wherein the glove is waterproof, thereby preventing bandages from getting wet while hand washing, showering and the like.

In one potential embodiment, the outer surface of the glove can be coated with a waterproof material. The use of a waterproof material, either coated or used as a layer in the glove, prevents melted snow, rain, water or moisture from penetrating through the glove to the hands and arms of the wearer, thus protecting the bandage and stitches of the wearer. The glove can be marketed as a part of the carpal tunnel surgery process and can be recommended by a doctor.

The post-op carpal tunnel surgery glove can be easily and independently placed on a wearer's hand by himself/herself without need of any assistance from another. Further, the glove can be used individually on either hand or in pairs, one on each hand, depending on the requirements of the wearer. The glove can have a anti-microbial layer to protect the wound and bandage in order to protect against cross-contamination. The gloves can be used by any adult or child and can help in reducing the healing time after undergoing carpal tunnel surgery without affecting the daily routine of the wearer.

The present invention provides a bandage covering of multiple layers which protects the bandage elements and bandage coverings from external or ambient liquids, while still providing a comfortable environment for the human wearer of the bandage. In accordance with the present invention, a bandage covering, adapted to protect a post-operative bandage from ambient dirt and liquids, is provided having an innermost layer including a soft hand feel, a waterproof and breathable middle layer permitting the passage of water vapor but preventing the passage of liquid water, and an outermost layer having a soft hand feel and abrasion resistance. Further in accordance with the invention, the bandage covering is washable in typical household-type washing machines and dryable in domestic dryers.

It is a further object of the present invention to provide a bandage covering which will isolate the liquids associated with normal wear of the bandage away from the bandage and skin of the wearer. It is another object of the present invention to provide a bandage covering which is sufficiently breathable to provide a comfortable and dry environment for an individual's post-operative stitches. It is still another object of the present invention to provide a bandage covering with an outer surface which can be dyed, patterned, and otherwise made visually pleasing in appearance. It is still another object of the present invention to provide a bandage covering which is waterproof, water vapor permeable, easy to manufacture, easy to cut to specific sizes and shapes, easy to decorate and machine washable/dryable.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective bottom and top view of a conventional post-operative carpal tunnel surgery bandage worn on the hand of an individual and over an incision and/or stitches;

FIG. 2 illustrates a perspective view of one potential embodiment of a post-operative carpal tunnel surgery glove of the present invention being worn by an individual after a carpal tunnel surgery in accordance with the disclosed architecture;

FIG. 3 illustrates a sectional view of one potential embodiment of the post-operative carpal tunnel surgery glove of the present invention in accordance with the disclosed architecture, wherein the different layers of the glove are displayed;

FIG. 4 illustrates a perspective view of one potential embodiment of a post-operative carpal tunnel surgery glove of the present invention being worn by an individual after a carpal tunnel surgery in accordance with the disclosed architecture, wherein the hook and loop fastener strap on the wrist is displayed;

FIG. 5 illustrates a sectional view of one potential embodiment of the hook and loop fastener of the post-operative carpal tunnel surgery glove of the present invention in accordance with the disclosed architecture, wherein the different layers of the fastener are displayed; and

FIG. 6 illustrates a perspective view of one potential embodiment of a post-operative carpal tunnel surgery glove of the present invention being worn by an individual after a carpal tunnel surgery in accordance with the disclosed architecture, wherein the waterproof nature of the glove is displayed.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention, nor do they limit the scope of the invention. Additionally, an illustrated embodiment need not have all the aspects or advantages shown. Thus, in other embodiments, any of the features described herein from different embodiments may be combined.

As noted above, there exists a long felt need in the art for a device to protect the post-operative bandage worn by individuals who have undergone carpal tunnel release surgery. There exists a long felt need in the art for a device to protect the post-operative bandage from getting wet while the individuals use water. There is also a long felt need in the art for a solution for covering post-operative bandages and keeping them dry, for example while showering. Additionally, there is a long felt need in the art for a cover for post-operative bandages which maximizes protection against water and cross-contamination of the bandage while hand washing, showering and the like. Further, there is a long felt need in the art for a cover for a post-operative bandage which keeps the bandage and stitches safe until the stitches are removed. Furthermore, there is a long felt need in the art for a cover for post-operative bandages that helps individuals to maintain a sanitary and dry bandage prior to removal of stitches. Finally, there is a long felt need in the art for a cover to be worn over post-operative bandages, which can be comfortably used and conveniently adjusted over the hands and wrist, as per the requirements of different wearers.

The innovative product of the present invention features a novel waterproof post-op carpal tunnel surgery glove structured to be worn after carpal tunnel surgery to prevent bandages and stitches from getting wet. The surgery glove comprises: an innermost layer, a middle layer, and an outermost layer; the innermost layer provides moisture permeability; the middle layer provides waterproofing capabilities; and the outermost layer provides dirt, dust and water repellency. The surgery glove is configured to be worn over a hand and covers the fingers, thumb, palm area and dorsal area while conforming to the wrist area of a wearer to protect the bandage covering a portion of the palm area and the wrist area after carpal tunnel surgery. The glove includes a hook and loop fastener strap for comfortable securement over the hand and the wrist.

The term “glove” is used herein in a broad sense to mean any flexible, sheet-like material that is passively and selectively held on a person's hand and covers a portion of the person's hand. Thus, the term glove can refer to a traditional glove which almost completely covers the hand and includes separate fingers, a mitt which almost completely covers the hand but does not have separate fingers, a portion of material which covers one side of the hand and is attached to the hand with an elastic band and which extends around the other side of the hand or any other portion of material that is passively held on the hand.

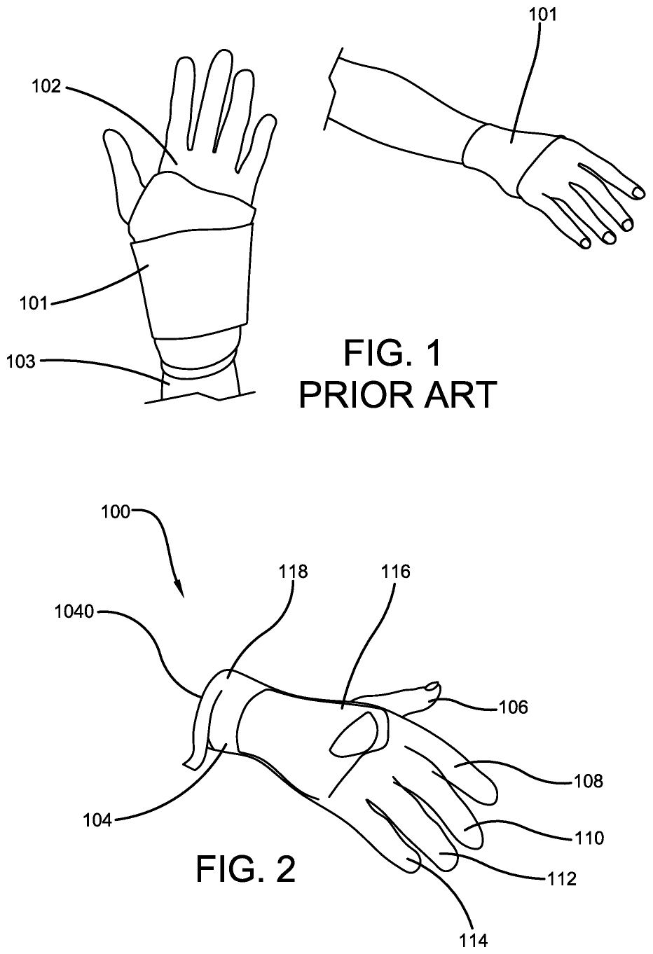

Referring initially to the drawings, FIG. 1 illustrates a perspective bottom and top view of a conventional post-operative carpal tunnel surgery bandage worn on the hand of an individual and over an incision and/or stitches. The carpal tunnel surgery is generally done to decompress the median nerve at the level of the wrist of a wearer to help relieve the pressure upon the median nerve. After a surgery, a soft dressing/bandage 101 covers the wound on the palm 102 and part of the forearm 103. However, the bandage 101 gets wet and contaminated easily from external or ambient water, dirt and moisture, which can cause infection and delay the healing process. Further, the wearer is prohibited from performing daily tasks effectively due to the fear of the bandage getting wet.

FIG. 2 illustrates a perspective view of one potential embodiment of a post-operative carpal tunnel surgery glove 100 of the present invention being worn by an individual after a carpal tunnel surgery in accordance with the disclosed architecture. The post-op carpal tunnel surgery glove 100 is waterproof, moisture permeable, lightweight, soft on the skin, and highly breathable. The glove 100 can be worn easily by a wearer to protect the post-operative bandage of carpal tunnel surgery. It should be appreciated that the glove 100 can protect any bandage worn over the palm area, wrist area, fingers or back area of the hand.

The post-op carpal tunnel surgery glove 100 comprises a wrist area 104 to cover the wrist area of a wearer. The wrist area 104 having an opening 1040 to slip on the post-op carpal tunnel surgery glove 100 onto a wearer's hand. The opening 1040 includes a hook and loop fastener 118 to secure the glove 100 over the wrist. The glove 100 includes a thumb portion 106, an index finger portion 108, a middle finger portion 110, a ring finger portion 1303 and a pinky finger portion 1302. The finger portions extend from the dorsal region cover 1301 and cover the corresponding fingers of the wearer. The wrist area 104 can secure tightly around a wearer's wrist using the hook and loop fastener 118. The glove 100 securely covers the palm area and the wrist area of the wearer's hand, along with the fingers and thumb from both the back area and the palm area to provide secure waterproofing and protection to the bandage worn over the wound due to the carpal tunnel surgery.

The glove 100 can be made to match size standards customary in the industry or be custom-made to fit a particular wearer's hand size. The glove 100 can be made to fit either the left hand or right hand. The glove 100 can be made of latex material, artificial fibers, general rubber, plastic membrane, nylon or any other suitable material that provides durability and flexibility for smooth operation of the glove 100. The glove 100 allows free movement of the fingers for making a first or stretching the fingers, while being smooth and soft to the skin.

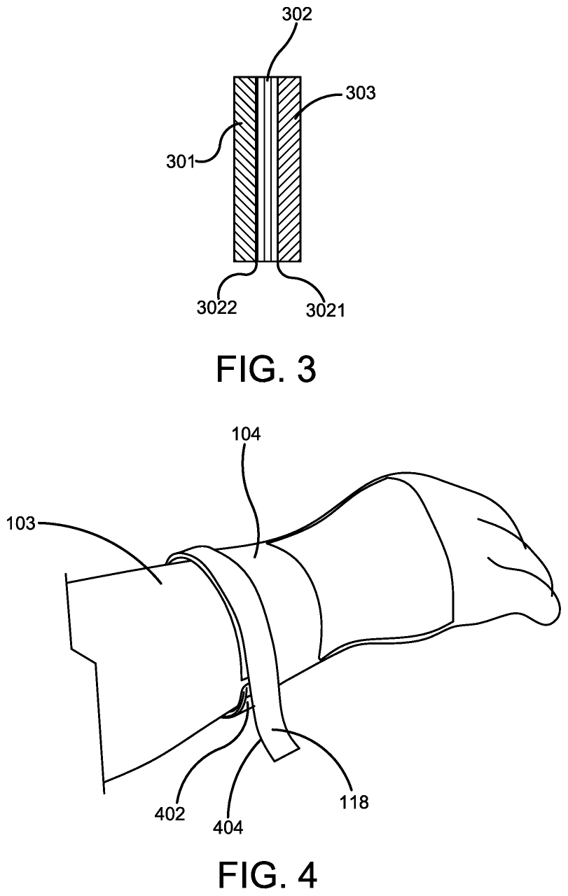

FIG. 3 illustrates a sectional view of one potential embodiment of the post-operative carpal tunnel surgery glove 100 of the present invention in accordance with the disclosed architecture, wherein the different layers of the glove are displayed. More specifically, the glove 100 is a multilayered glove having a plurality of functional layers. The innermost layer 301 touches the skin of a wearer and is soft on the skin. The innermost layer 301 provides moisture permeability, allowing the discharge of sweat from a wearer's skin to other outer layers. The innermost layer 301 is stretchable, such that a wearer can easily wear it over a bandage without any discomfort. The innermost layer can be a fabric layer. To be described hereinafter, the innermost layer includes a different composition than the other layers. In one potential embodiment, the innermost layer 301 is a cotton-polyester blend which provides water vapor permeability and good hand-feel.

The middle layer 302 includes a waterproofing layer. The middle layer 302 simultaneously allows sweat, from a wearer of the glove 100, to travel away from a wearer's hand but does not allow the outside moisture and water to travel inside to the innermost layer 301. The middle layer 302 is formed of stretchable water absorption prevention fabric. The middle layer 302 is further formed of stretchable waterproof and breathable material. It is to be appreciated that the stretchable waterproof and breathable middle layer includes an inner face 3022 adjacent the innermost layer 301, and an outer face 3021 adjacent an outermost layer 303. The middle layer 302 simultaneously permits the passage of water vapor from the inner face 3022 to the outer face 3021 in a first direction, while preventing the passage of liquid water from the outer face 3021 to the inner face 3022 in a second direction. In this manner, the middle layer 302 prevents the flow of water from its outer face 3021 through its body to its inner face 3022. The middle layer 302 allows the flow of water vapor from the outer face 3021 through the body of the layer to the inner face 3022, and also allows the flow of water vapor from the inner face 3022 to the outer face 3021. Several different materials are available having these characteristics. However, the constraints of making the finished product home machine washable and dryable, manufacturable by lamination and sufficiently breathable and waterproof to form a bandage covering are significant.

In one potential embodiment, stretchable water vapor permeable synthetic polymer film materials, preferably a fluoropolymer film, are appropriate for use as the middle layer 302. An expanded polytetrafluoroethylene film is most preferable. Alternative materials for use in the middle layer 302 include stretchable polymer-encapsulated cotton, wax-impregnated closely woven cotton or cotton-polyester mixes, or other fabrics or membranes allowing the passage of water vapor but preventing the passage of liquid water.

The outermost layer 303 provides dirt, dust, and water repellency. The outermost layer 303 can be of any color and structure. The outermost layer 303 can be attached to the middle layer 302 through an adhesive. The outermost layer 303 can be a water-repellant material to resist liquid water from penetrating therethrough. The outermost layer 303 can be a stretchable plain weave construction, resistant to snagging, while also providing good hand-feel and washability. The outermost layer 303 allows the passage of water vapor therethrough from the layers below. A plain weave can be created by having fill thread go over one weft thread and under the next thread repeatedly. The next fill thread goes over the weft threads its neighbor went under, and under the weft threads its neighbor went over. A simple, tight weave can be achieved which aids in snag resistance. A high thread count also aids in achieving a tight weave which provides snag and puncture resistance.

It should be appreciated that the number of layers can be more or less depending upon the requirements of the wearers. However, in all embodiments, the glove 100 provides waterproofing capabilities to protect the post op carpal surgery bandage worn by the wearer. The innermost, middle and outermost layers together comprise a thickness to provide for both tactile sensitivity and durability. The thickness is generally from about 6 mils to about 14 mils.

By wearing the glove 100, it is possible to block the penetration of water from the elements outside of the bandage, and also to quickly discharge the internal moisture from the skin and bandage to the outside, to create an optimal moisture-free environment to heal the wound and maintain longevity of the bandage. The outermost layer 303, the middle layer 302, and the innermost layer 301 are laminated together by laminating glue, adhesive or other conventional means. The outermost layer 303 is normally dyed or printed to provide a pleasing appearance. The outermost layer 303 and the innermost layer 301 protect the middle layer 302 from abrasion, piercing or snagging. It is to be appreciated that the human wearer of the glove 100 needs an environment that allows the passage of water vapor away from the individual's skin. The glove 100 of the present invention allows for the passage of water vapor through the layers 301, 302, 303 so that a dry environment is provided to the bandages below. When the glove 100 becomes soiled, wet or discolored, it can be removed from the person's hand and washed in a regular household water-based washing machine. Following washing, the glove 100 is machine dryable.

FIG. 4 illustrates a perspective view of one potential embodiment of a post-operative carpal tunnel surgery glove 100 of the present invention being worn by an individual after a carpal tunnel surgery in accordance with the disclosed architecture, wherein the hook and loop fastener strap 118 on the wrist is displayed. More specifically, to provide a secure fit around the arm/wrist 103 of a wearer, the wrist area 104 of the glove 100 includes a hook and loop fastener strap 118 having a loop fastener 402 and a hook fastener 404. In one potential embodiment, the wrist area 104 can include a VELCRO® Brand Double-Faced Loop, or a hook and loop fastener, having a breathable, flexible, and extra soft loop featuring an embossed pattern on the surface. The hook and loop strap can be adjusted to conform to a wearer's arm, thus increasing comfort. Further, the hook and loop strap does not cause skin irritation and includes superior breathability.

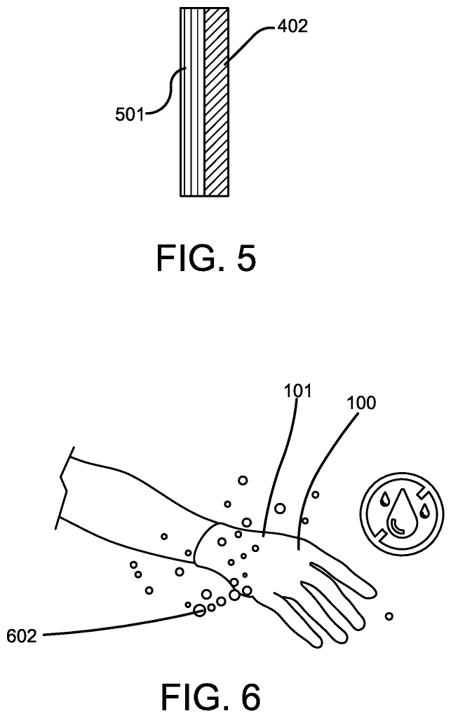

FIG. 5 illustrates a sectional view of one potential embodiment of the hook and loop fastener 118 of the post-operative carpal tunnel surgery glove 100 of the present invention in accordance with the disclosed architecture, wherein the different layers of the fastener 118 are displayed. More specifically, to provide comfort to a patient, the glove 100 around the wrist area includes a bottom lining 501 of linen, cotton or polyester. The bottom lining 501 conforms and secures to the arm of the wearer/patient. A second layer of loop fastener 402 is attached to the bottom lining 501 and the hook fastener 404 removably attaches to the loop fastener 402.

FIG. 6 illustrates a perspective view of one potential embodiment of a post-operative carpal tunnel surgery glove 100 of the present invention being worn by an individual after a carpal tunnel surgery in accordance with the disclosed architecture, wherein the waterproof nature of the glove 100 is displayed. As previously disclosed, the post-op carpal tunnel surgery glove 100 comprises a waterproof glove worn generally after carpal tunnel surgery to protect the bandage 101 from ambient or external water and moisture 602. The glove 100 can prevent the bandage 101 and stitches from getting wet for an extended period of time, for example up to the time of stitch removal. The glove keeps the bandage 101 dry at all times. The glove 100 maximizes protection against water and cross-contamination of the bandage 101 while the wearer engages in hand washing, showering and the like.

The dorsal region 1301 can have any logo, embroidery, structure, theme, picture and the like to make the glove 100 more aesthetically pleasing while maintaining the waterproofing capabilities. The glove 100 can be available in various sizes, structures and colors. The glove 100 can be worn by men, women and children to effectively protect the bandages. The glove 100 of the present invention is durable and long-lasting, can be made in various sizes, of various materials and colors and is cost-effective from both a manufacturer's and consumer's point of view.

It is yet another feature of the glove 100 of the present invention that, by virtue of the softness and flexibility of the material, the wearer can perform necessary hand and finger exercises. Further, the glove 100 is readily foldable, and is readily and conveniently transportable. The glove 100 includes medical-grade material that is resistant to tearing and provides comfort from post-operative pain to the wearer of the glove. The glove 100 is nitrile-free. The glove 100 can include good tactile sensitivity. Each glove 100 is sterile when packaged and includes superior fit and comfort. The glove 100 further offers less hand fatigue and stress. The gloves 100 can comprise polyvinylchloride (PVC) materials free, from latex and nitrile.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “post-op carpal tunnel surgery glove”, “waterproof glove”, “glove”, “bandage protective gloves”, and “hand glove” are interchangeable and refer to the post-op carpal tunnel surgery glove 100 of the present invention.

Notwithstanding the forgoing, the post-op carpal tunnel surgery glove 100 of the present invention can be of any suitable size and configuration as is known in the art without affecting the overall concept of the invention, provided that it accomplishes the above-stated objectives. One of ordinary skill in the art will appreciate that the size, configuration and material of the post-op carpal tunnel surgery glove 100 of as shown in the FIGS. are for illustrative purposes only, and that many other sizes of the post-op carpal tunnel surgery glove 100 are well within the scope of the present disclosure. Although the dimensions of the post-op carpal tunnel surgery glove 100 are of important structure parameters for wearer convenience, the post-op carpal tunnel surgery glove 100 can be of any size that ensures optimal performance during use and/or that suits wearer needs and/or preferences.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A bandage covering for covering a post-operative hand bandage adapted to protect the post-operative hand bandage and stitches thereunder, said bandage covering comprising:
a stretchable innermost layer being at least partially cotton;
a stretchable waterproof and breathable middle layer having an inner face adjacent said stretchable innermost layer and an outer face adjacent an outermost layer, said middle layer simultaneously permitting the passage of water vapor from said inner face to said outer face in a first direction, while preventing the passage of liquid water from said outer face to said inner face in a second direction;
said middle layer is a fluoropolymer layer;
said outermost layer is a stretchable water and dirt repelling outermost layer at least partially cotton for providing wear resistance to said outermost layer;
said stretchable innermost layer, said middle layer, and said outermost layer formed into a finger portion, a palm portion, a dorsal portion, and a wrist portion for encapsulating respective hand sections of a wearer;
said wrist portion having a hook and loop fastener strap for wrapping and securing to the wearer's wrist; and
wherein said stretchable innermost layer, said middle layer, and said outermost layer permit said passage of said water vapor in said first direction, traveling from the wearer between said post-operative bandage and said bandage covering, through first said stretchable innermost layer, then said middle layer, and finally said outermost layer.

2. The bandage covering of claim 1, wherein said stretchable innermost layer, said middle layer, and said outermost layer together comprise a thickness for tactile sensitivity and durability, said thickness is generally from about 6 mils to about 14 mils.

3. The bandage covering of claim 2, wherein said thickness is generally from about 8 mils to about 12 mils.

4. The bandage covering of claim 3, wherein said outermost layer is a plain weave fabric.

5. The bandage covering of claim 4, wherein said outermost layer is puncture resistant.

6. The bandage covering of claim 5, wherein said middle layer is a polytetrafluoroethylene layer.

7. The bandage covering of claim 6 wherein said middle layer is an expanded polytetrafluoroethylene layer.

8. A waterproof, water-vapor-permeable bandage covering for covering a post-operative bandage adapted to protect the post-operative bandage from ambient liquids, said bandage covering comprising:
a stretchable innermost layer being at least partially cotton;
a stretchable waterproof and breathable middle layer having an inner face adjacent said innermost layer and an outer face adjacent an outermost layer, said middle layer simultaneously permitting the passage of water vapor from said inner face to said outer face in a first direction, while preventing the passage of liquid water from said outer face to said inner face in a second direction;
said middle layer is a polytetrafluoroethylene layer;
said outermost layer is a stretchable water and dirt repelling outermost layer at least partially cotton for providing wear resistance to said outermost layer;
said innermost layer, said middle layer, and said outermost layer formed into a finger portion, a palm portion, a dorsal portion, and a wrist portion for encapsulating respective hand sections of a wearer;
said wrist portion having a hook and loop fastener strap for wrapping and securing to the wearer's wrist;
wherein said innermost layer, said middle layer, and said outermost layer permit said passage of said water vapor, in said first direction, traveling from the wearer between said post-operative bandage and said bandage covering, through first said innermost layer, then said middle layer, and finally said outermost layer; and
said bandage covering being washable in a water based domestic home type washing machine.

9. The bandage covering of claim 8, wherein said innermost layer, said middle, and said outermost layer together comprise a thickness for tactile sensitivity and durability, said thickness is generally from about 6 mils to about 14 mils.

10. The bandage covering of claim 8, wherein said thickness is generally from about 8 mils to about 12 mils.

11. The bandage covering of claim 10, wherein said outermost layer is a plain weave fabric.

12. The bandage covering of claim 11, wherein said outermost layer is puncture resistant.

13. The bandage covering of claim 12, wherein said middle layer is an expanded polytetrafluoroethylene layer.

14. A waterproof, water-vapor-permeable bandage covering for covering a post-operative bandage adapted to protect the post-operative bandage from ambient liquids, said bandage covering comprising:
a stretchable innermost layer being at least partially cotton;
a stretchable waterproof and breathable middle layer having an inner face adjacent said innermost layer and an outer face adjacent an outermost layer, said middle layer simultaneously permitting the passage of water vapor from said inner face to said outer face in a first direction, while preventing the passage of liquid water from said outer face to said inner face in a second direction;
said outermost layer is a stretchable water and dirt repelling outermost layer at least partially cotton for providing wear resistance to said outermost layer;
said innermost layer, said middle layer, and said outermost layer formed into a finger portion, a palm portion, a dorsal portion, and a wrist portion for encapsulating respective hand sections of a wearer;
wherein said innermost layer, said middle layer, and said outermost layer permit said passage of said water vapor, in said first direction, traveling from the wearer between said post-operative bandage and said bandage covering, through first said innermost layer, then said middle layer, and finally said outermost layer; and
said bandage covering being washable in a water based domestic home type washing machine.

15. The bandage covering of claim 14, wherein said innermost layer, said middle, and said outermost layer together comprise a thickness for tactile sensitivity and durability, said thickness is generally from about 6 mils to about 14 mils.

16. The bandage covering of claim 15, wherein said wrist portion having a hook and loop fastener strap for wrapping and securing to the wearer's wrist.

17. The bandage covering of claim 16, wherein said outermost layer is a plain weave fabric.

18. The bandage covering of claim 17, wherein said outermost layer is puncture resistant.

19. The bandage covering of claim 18, wherein said middle layer is an expanded polytetrafluoroethylene layer.

20. The bandage covering of claim 14 further comprising a thickness that is generally from about 8 mils to about 12 mils.
